(19) United States
(12) Patent Application Publication
(10) Pub. No.: US 2026/0234522 A1
(43) Pub. Date: Aug. 13, 2026

(54) Cultureware Slope Features for Seed Cell Concentration

(71) Applicant: XDemics Corporation, Novato, CA (US)

(72) Inventors: Colin A. Cook, Oakland, CA (US); Nicholas E. Scianmarello, San Francisco, CA (US)

(73) Assignee: XDemics Corporation, Novato, CA (US)

(21) Appl. No.: 19/536,259

(22) Filed: Feb. 11, 2026

Related U.S. Application Data
(60) Provisional application No. 63/757,456, filed on Feb. 12, 2025.
(60) Provisional application No. 63/770,927, filed on Mar. 12, 2025.
(60) Provisional application No. 63/874,207, filed on Sep. 2, 2025.

(51) Int. Cl. C12M 1/24; C12M 1/00; C12M 1/32; H10D 30/01

(52) U.S. Cl. CPC C12M 23/08; C12M 23/12; C12M 23/34; H10D 30/024

(57) ABSTRACT

Cultureware, such as petri dishes, well plates, tubes, and flasks, are made with a gas permeable bottom having rows of tiny fins configured to trap biological cells in the grooves between them. The tops of the fins can be rounded or slanted such that seed cells that are deposited into the cultureware roll into the grooves. The fins are parallel to each other but can be formed in a wavy, zig-zag, or other undulating pattern. The undulating fin cultureware can be rested on a slanted surface such that deposited seed cells roll into dips formed by the undulations. Footings underneath the resilient bottom that run orthogonal to the grooves can, during centrifugation, promote the formation of temporary local minima in the unsupported areas between the grooves. Seeded cells that are deposited into the cultureware will roll into the local minima when centrifuged and stay congregated with each other when the cultureware is removed from the centrifuge. Devices, methods of manufacture, and use of cultureware with such features are disclosed.

### CROSS-REFERENCES TO RELATED APPLICATIONS

This application claims the benefit of U.S. Provisional Patent Application No. 63/757,456, filed 12 Feb. 2025, U.S. Provisional Patent Application No. 63/770,927, filed 12 Mar. 2025, and U.S. Provisional Patent Application No. 63/874,207, filed 2 Sep. 2025, the contents of which are hereby incorporated by reference in their entireties for all purposes.

### STATEMENT AS TO RIGHTS TO INVENTIONS MADE UNDER FEDERALLY SPONSORED RESEARCH AND DEVELOPMENT

Not applicable.

### BACKGROUND

### 1. Field of the Invention

The present application generally relates to containers or dishes for laboratory use in retaining material to be analyzed with fluid transport features. Specifically, the application is related to petri dishes, single-and multi-well plates, and other cultureware with elongated bottom fin features that have rounded or slanted tops such that biological cells fall off into grooves below and/or fins and grooves with permanent or temporary depressions for concentrating cells together.

### 2. Description of the Related Art

Cultureware, such as petri dishes, well plates, tubes, and flasks, are used to grow cell cultures. The devices are seeded with relatively few cells and fed with a liquid or gel culture medium. With time, the cells divide and expand into colonies of cells.

Cultureware's use is a relatively inexpensive and straightforward way to increasing the number and volume of cells with which to study, diagnose, or employ for therapy. However, there can be issues when starting with very few cells, and the issues occur across a wide number of applications within biology and medicine.

Low seeding densities or low viability cells can result in much delayed—or abandoned—cell cultures. The cells just cannot multiply fast enough. The time and growth must be recovered. In an extreme example of clonal cell line development, a single cell acts as the source of the entire population and must recover from 1 cell per well. In cell therapy applications, such as Chimeric Antigen Receptor T-cell (CAR-T) production, the starting cells may be in poor condition or viability due to stress of the disease and chemotherapies a patient is undergoing or has undergone.

Many cell types experience a significant reduction in proliferation when they are seeded at low density, sometimes referred to as a “lag phase.” There are several known factors that contribute to this. Autocrine and paracrine signals, such as growth factors, cytokines, lipid mediators, Wnt (Wingless-related iNTegration site) proteins, and exosomes, can induce cells into a proliferative and/or pro-survival state. When the concentration of cells in a culture vessel is low, levels of these signals are also low. When signals are low, cells may not proliferate, and they may lose viability. Juxtacrine signaling, such as notch signaling, can also induce cells into a proliferative and/or pro-survival state through physical contact. When cell density is low, cells may not find themselves in physical or chemical contact with neighboring cells. Environmental stressors can also negatively impact viability and proliferation. This can include insufficient oxygenation or CO₂ exchange, sudden temperature shifts, osmotic changes, and shear stresses. Thus, cells tend to grow and multiply better when concentrated with each other.

U.S. Pat. No. 11,981,885 discloses cultureware with a gas-permeable membrane on the bottom that has elongated surface features that physically protect cells and channel oxygen to them. The elongated surface features form long niches where cells grow.

In bioproduction and laboratory applications, there is a need to maximize the dynamic range of cell growth expansion, i.e., grow cells equally satisfactorily whether there are just a few cells or millions of cells. This would help minimize the amount of touch time or number of stages in a seed train.

### BRIEF SUMMARY

Generally, laboratory cultureware is described that includes elongated, gas-permeable fin features along the bottom of the cultureware vessel's liquid holding area that are rounded or slanted on the top. Between the fin features are grooves in which biological cells are protected and can grow and multiply. The rounded or slanted tops of the fins facilitate cells falling off the tops and settling into the grooves. The bottoms of the grooves can include lower points than others, called depressions, in which cells can further fall and naturally pool together.

Footings on the underside of resilient cultureware can be spaced so that—upon centrifuging—the resilient bottoms of the grooves deform downward into temporary depressions, minima, when the cultureware. Any seeded cells in the grooves will roll or fall into the depressions and thus gather together. When removed from the centrifuge, the cells remain fairly close to where they gathered.

As an alternative or in addition to having straight fins, cultureware can employ fins that are formed in a wavy or zig-zag pattern, or otherwise undulate laterally. The undulations can be repeated every few millimeters, for example. When the cultureware is tilted upward, seed cells can drain from the fin tops and grooves into low points of the undulations. When re-righted, the cultureware is left with concentrations of cells in concave portions of the undulations.

Methods of manufacture and use of the cultureware are also described.

Some embodiments of the present invention are related to a cultureware apparatus including a basin base, a sidewall surrounding the basin base, and parallel fins projecting upward from the basin base and defining grooves therebetween configured to confine biological cells, wherein the fins and base comprise a gas-permeable material, and wherein a cross section of each fin of the fins has a rounded or slanted top.

A lateral cross section of each fin can be symmetric around a vertical line. Each top can be entirely slanted or entirely rounded. An edge of each top can be chamfered or filleted. A pair of adjacent fins can each have a top in which more than a half of the respective top slopes toward a groove shared between the pair.

The parallel fins can extend short of the sidewall to form a perimeter groove, sometimes referred to as a peripheral groove, along the sidewall that surrounds the fins.

The parallel fins can be in rows that undulate laterally. The undulating rows can be in a wavy pattern or a zig-zag pattern. Inflection points of the zig-zag pattern can be rounded. Each fin in the undulating rows can be symmetric about an average centerline.

A cultureware assembly can include a pedestal wedge with the cultureware supported underneath by the pedestal wedge with an average centerline of the rows extending non-parallel to a slope of the wedge. Non-parallel can include perpendicular.

The basin base can be transparent, each groove of the grooves can have a flat bottom, and a thickness of the basin base directly underneath each groove can be constant along a length of the groove. This can be helpful for imaging, where light passes through the base.

The basin base can include a depression within a groove of the grooves. The depression can narrow to a point. The depression can include a trench.

The cultureware can include elongated footings projecting downward from the basin base, each footing of the footings extending laterally with respect to the fins. The basin base can include a depression within a groove of the grooves, the depression centered between a pair of the footings.

At least one of the fins can have a height between 400 μm (microns) and 1000 μm, a width between 300 μm and 500 μm, and a pitch between 200 μm and 500 μm. The gas-permeable material can be permeable to diatomic oxygen (i.e., O₂) or carbon dioxide (i.e., CO₂). Biological cells have a diameter on the order of 20 μm.

Some embodiments are related to a cultureware apparatus including a basin base, a sidewall surrounding the basin base, and parallel fins projecting upward from the basin base and defining grooves therebetween configured to confine biological cells, wherein the fins and base comprise a gas-permeable material, wherein the parallel fins are in rows that undulate laterally.

The undulating rows can be in a wavy, sinusoidal-like curve or a straighter zig-zag pattern. Inflection points of the zig-zag pattern can be rounded. Each fin of the undulating rows can be symmetric about an average centerline.

A cultureware assembly can include a pedestal wedge with the cultureware supported underneath by the wedge with an average centerline of the rows extending non-parallel to a slope of the wedge.

Biological cells can exist within the grooves and concentrated in concave portions of the undulating rows within the grooves.

Some embodiments are related to a cultureware apparatus including a basin base comprised of a resilient material, a sidewall surrounding the basin base, parallel fins projecting upward from the basin base and defining grooves therebetween configured to confine biological cells, and elongated footings projecting downward from the basin base, each footing of the footings extending laterally with respect to the fins, wherein the fins and base comprise a gas-permeable material.

The basin base can include a depression within a groove of the grooves, the depression centered between a pair of the footings.

A centrifuge cultureware assembly can include a centrifuge plate experiencing a g-force equal-to-or-greater-than 10 times a force of gravity with the cultureware supported by the footings on the centrifuge plate, wherein basin base portions that are under the grooves and between pairs of the footings are deformed toward the centrifuge plate, thereby creating local minima. Biological cells can exist within the grooves and be concentrated in the local minima.

Some embodiments are related to a cultureware apparatus including a basin base, a sidewall surrounding the basin base, and parallel fins projecting upward from the basin base and defining grooves therebetween configured to confine biological cells, wherein the fins and base comprise a gas-permeable material, wherein the parallel fins extend short of the sidewall to form a perimeter groove along the sidewall that surrounds the fins.

Each fin of the fins can have a rounded or slanted top. A depth of the perimeter groove can be at a same depth as a depth of the grooves.

Some embodiments are related to a method of manufacturing cultureware with rounded- or slanted-top fins, the method including forming a basin base, forming a sidewall surrounding the basin base, and forming parallel fins projecting upward from the basin base to define grooves therebetween that are configured to confine biological cells, wherein the fins and base comprise a gas-permeable material, wherein a cross section of each fin of the fins has a rounded or slanted top.

Some embodiments are related to a method of manufacturing cultureware with undulating cell niches, the method including forming a basin base, forming a sidewall surrounding the basin base, and forming parallel fins projecting upward from the basin base to define grooves therebetween configured to confine biological cells, wherein the fins and base comprise a gas-permeable material, wherein the parallel fins are in rows that undulate laterally.

Some embodiments are related to a method of manufacturing cultureware with elongated footings, the method including forming a basin base comprised of a resilient material, forming a sidewall surrounding the basin base, and forming parallel fins projecting upward from the basin base to define grooves therebetween that are configured to confine biological cells, and forming elongated footings projecting downward from the basin base, each footing of the footings extending laterally with respect to the fins, wherein the fins and base comprise a gas-permeable material.

Some embodiments are related to a method of manufacturing cultureware with a perimeter groove, the method including forming a basin base, forming a sidewall surrounding the basin base, and forming parallel fins projecting upward from the basin base to define grooves therebetween that are configured to confine biological cells, wherein the fins and base comprise a gas-permeable material, wherein the parallel fins extend short of the sidewall to form a perimeter groove along the sidewall that surrounds the fins.

Some embodiments are related to a method of using cultureware, the method including providing a cultureware with a basin base, a sidewall surrounding the basin base, and parallel fins projecting upward from the basin base to define grooves therebetween that are configured to confine biological cells, wherein the fins and base comprise a gas-permeable material, wherein a cross section of each fin of the fins has a rounded or slanted top, dispensing a liquid carrying biological cells into the cultureware, and jiggling the cultureware or agitating the liquid in order to allow a cell of the biological cells that has settled on a fin to slide off the rounded or slanted top of the fin into a groove of the grooves.

A pair of adjacent fins each can have a top in which more than a half of the respective top slopes toward a groove shared between the pair. The basin base can be transparent, each groove of the grooves can have a flat bottom, and a thickness of the basin base directly underneath each groove can be constant along a length of the groove. The method can further include projecting light through the basin base and imaging cells within the grooves.

Some embodiments are related to a method of using cultureware to concentrate seed cells, the method including providing a cultureware with a basin base, a sidewall surrounding the basin base, and parallel fins projecting upward from the basin base to define grooves therebetween that are configured to confine biological cells, wherein the fins and base comprise a gas-permeable material, wherein the parallel fins are in rows that undulate laterally, dispensing a liquid carrying biological cells into the cultureware, tilting the cultureware, and allowing the biological cells to concentrate in concave portions of the undulating rows within the grooves.

The method can further include untilting (i.e., righting with respect to gravity) the cultureware, and then culturing the concentrated cells.

Some embodiments are related to a method of using cultureware to concentrate seed cells, the method including providing a cultureware with a basin base comprised of a resilient material, a sidewall surrounding the basin base, parallel fins projecting upward from the basin base to define grooves therebetween that are configured to confine biological cells, and elongated footings projecting downward from the basin base, each footing of the footings extending laterally with respect to the fins, wherein the fins and base comprise a gas-permeable material, dispensing a liquid carrying biological cells into the cultureware, centrifuging the cultureware to deform basin base portions that are under the grooves and between pairs of the footings, thereby creating local minima, and allowing the biological cells to concentrate in the local minima.

The method can further include removing the cultureware from a centrifuge machine and culturing the concentrated cells.

### BRIEF DESCRIPTION OF THE DRAWINGS

FIG. 1A is a top-side perspective view of a one-well cultureware vessel as well as a close up and geometry legend in accordance with an embodiment.

FIG. 1B is a bottom-side perspective view of the one-well cultureware of FIG. 1A.

FIG. 2 is a vertical cross section of rounded-top fins in accordance with an embodiment.

FIG. 3 is a vertical cross section of pointed-top fins in accordance with an embodiment.

FIG. 4 is a vertical cross section of radius-cornered top fins in accordance with an embodiment.

FIG. 5 is a vertical cross section of chamfered-and rounded-top fins in accordance with an embodiment.

FIG. 6 is a vertical cross section of triangular fins in accordance with an embodiment.

FIG. 7 is a vertical cross section of rounded-top fins with pointed bottom grooves in accordance with an embodiment.

FIG. 8 is a vertical cross section of pairs of fins slanted into common grooves in accordance with an embodiment.

FIG. 9 is a vertical cross section of several types of groove bottoms in accordance with an embodiment.

FIG. 10A is a top down view of a shallow diamond point groove depression in accordance with an embodiment.

FIG. 10B is a vertical cross section of a shallow diamond point groove depression in accordance with an embodiment.

FIG. 11A is a vertical cross section view of a groove in accordance with an embodiment.

FIG. 11B is a vertical cross section view of a resilient groove undergoing centrifugation in accordance with an embodiment.

FIG. 11C is a vertical cross section view of a post-deformed groove in accordance with an embodiment.

FIG. 12 is a top-side perspective view of wavy rows of fins in accordance with an embodiment.

FIG. 13 is a top view of a zig-zag pattern of rows of fins in accordance with an embodiment.

FIG. 14 is a perspective view of a laterally undulating fin cultureware dish on a pedestal wedge in accordance with an embodiment.

FIG. 15 is a flowchart illustrating a process in accordance with an embodiment.

FIG. 16 is a flowchart illustrating a process in accordance with an embodiment.

FIG. 17 is a flowchart illustrating a process in accordance with an embodiment.

FIG. 18 is a flowchart illustrating a process in accordance with an embodiment.

### DETAILED DESCRIPTION

High density cell respirator (HDCR) technology can include gas permeable protrusions in the inside bottom of cultureware to control a cell expansion niche microenvironment during cell culture. Such cultureware can include petri dishes, single-and multi-well plates, flasks, and other items for culturing biological microorganisms, such as bacteria, yeasts, and molds. The protrusions can carry out several functions. One function is to enhance oxygen delivery. Another is to protect cells from shear forces and disruption during media exchange and manipulations.

The cells that are cultured can be for the production of biological products, such as proteins and viruses. For example, such cell lines can include: Chinese hamster ovary (CHO), human embryonic kidney (HEK)293, Spodoptera frugiperda (SF)9, A549 adenocarcinomic human alveolar basal epithelial cells, Henrietta Lacks (HeLa), Vero African green monkey, baby hamster kidney (BHK), human cell line PER.C6, Madin-Darby canine kidney (MDCK), hybridomas, bacterial cells, yeast cells, plant cells, fungal cells, insect cells and immortalized cell lines.

In addition to protecting cells, the protrusions and niche geometry can be engineered to concentrate cells and create a stagnant zone to promote autocrine and paracrine concentration. The combination of these features, among others, can allow for the efficient expansion of cells from ultra-low seeding densities as well as recovery from poor cell viability.

A technical advantage in concentrating cells together is in increasing local autocrine, paracrine, and juxtacrine signaling. This can help to reduce lag phase, induce proliferation, and promote cell survival.

Elongated protrusions (e.g., fins) extending upward from the internal bottom of cultureware can be sloped or convex to shed cells, prevent them from settling on top of them during seeding. This results in more cells concentrating in the groove niches between them.

The slope of the top of the protrusions can all be in the same direction in order to achieve a uniform concentration of cells into niches. Alternatively, the tops of the protrusions can be a peaked in shape or a convex shape (e.g. rounded, ellipsoidal, curved). The shape can be symmetric to achieve a uniform concentration of cells. Alternatively, the slopes of the protrusions can alternate direction, resulting in a more significant concentration of cells into every other set of niches. Such a result can also be achieved by using a non-symmetric shape for the top of the elongated protrusions.

While seeding, cultureware can be placed on a slope to prevent cells from settling on flat-topped protrusions. Yet there may be further advantages with certain features. Placing the cultureware on a slope can cause cells to collect against the edge of a protrusion, thereby increasing local cell concentration. For example, in the case of cultureware with rows of protrusions (e.g. fins) one could tilt the plate perpendicular to the direction of the rows (i.e. each row is at a different height than the other rows).

To further increase cell concentration, the media can be gently agitated, such as by jiggling the cultureware, to sweep cells on the top of protrusions off and down into the niches. Agitation should be mild enough so as not to fluidize cells from the niches. For example, fluid could be passed perpendicularly over the protrusions such as by rocking or pipetting. Alternatively, the device can be vibrated to induce cell settling into place.

Cells can be removed from flat-topped protrusions by centrifugation of the cultureware on a sloped wedge insert. The insert can be separate or integral with the cultureware.

Rows of protrusions can be engineered as wavy, zig-zag, or otherwise undulating to create local minima for cell settling and concentration when the device is seeded on an angle.

Spacing features, such as pillars, posts, fins, protrusions, or mesh, underneath the membrane can induce curvature of the membrane under the weight of the device itself, the weight of media, the force of centrifugation, or pressure. This can provide predictable and engineerable minima for cells to settle into and concentrate. In the case of centrifugation, the generation of the minima can be a transient phenomenon that brings cells into proximity to one another. Upon removal from the centrifugal force, the membrane may restore to its initial shape—but the cells can remain in close proximity due to friction, stickiness, and protrusions on the cell-side of the membrane.

The bottom of each niche or groove can be sloped, troughed, rounded, cusped, or otherwise concave in shape to locally concentrate cells. This feature can be in one dimension (1D), such as to concentrate cells along a line, or in two dimensions (2D) such as to concentrate cells into a spot or area.

A technical advantage in flanking the cell expansion niche with protrusions, such as rows of fins, is to help to enhance autocrine and paracrine signaling. Firstly, they can locally concentrate cells such that the levels of these signals will be higher. Secondly, the protrusions can restrict the ability of the signals to diffuse away, leading to higher concentrations of the signals. Thirdly, the protrusions can create a stagnant fluid microenvironment that prevents the signals from being carried away by flow, mixing, or turbulence.

A technical advantage in fabricating the cultureware from gas permeable material is in that it decouples gas exchange from the soluble nutrient reservoir. This allows a large volume of cell culture media to overlay the cells without compromising oxygen supply. A larger reservoir of media can contribute to greater environmental stability by increasing the thermal mass of the system to reduce the rate of change in temperature. It similarly increases the total buffering capacity of the system with respect to CO₂ or pH. Larger reservoirs of media can experience slower changes in osmotic pressure (e.g. such as from evaporation). Protrusions in the cultureware can create an insulated cell expansion niche that protects cells from shear forces.

Cell recovery and proliferation can be enhanced further through the introduction of supplements into the media. These supplements can include insulin, transferrin, selenium (ITS) mix, carrier proteins (e.g. albumin), recombinant growth factors (e.g., Epidermal Growth Factor (EGF), Fibroblast Growth Factor Family (FGF), Insulin-like Growth Factor (IGF), Transforming Growth Factor (TGF)-β for specific cell types), and Rho-associated protein kinase (ROCK) inhibitor (e.g., Y-27632) for stem cells and epithelial lines (e.g. to promote survival post-dissociation). Conditioned medium (e.g. from log-phase cultures of the same cell line) can be used or supplemented to boost secreted growth factors, extracellular matrix components, and survival signals. Finally, feeder cells can be added to provide paracrine support (e.g. co-culture with irradiated fibroblasts or mesenchymal stem cells).

A peripheral or common groove/channel, which connects a plurality or all cell expansion niches/grooves, can improve the uniformity of cell seeding and expansion within the device by facilitating redistribution of cells either fluidically or via migration. The peripheral or common groove/channel can also improve the distribution of nutrients to cells by increasing the fluid flow path between grooves. The peripheral or common groove/channel can also facilitate the removal of metabolites, cellular byproducts, or other waste materials away from cells by increasing the fluid flow path between grooves.

The peripheral channel in the form of a perimeter groove, which surrounds the rows of grooves, can have a bottom that is higher, lower, or at the same depth as that of the grooves.

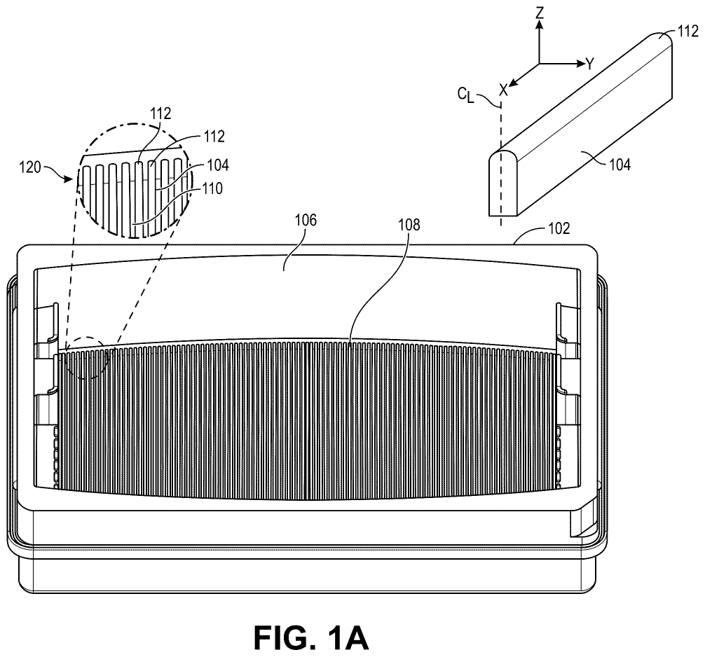

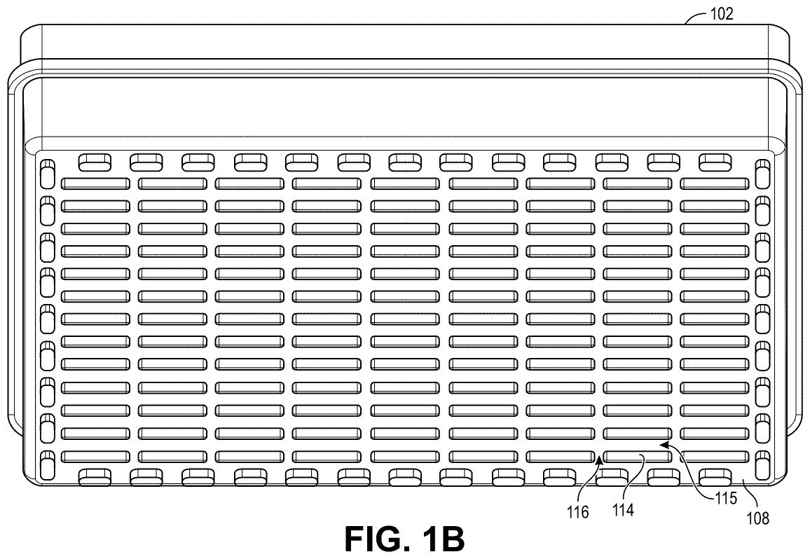

FIGS. 1A-1B illustrates a one-well cultureware vessel 102. Cultureware 102 includes thin polymer membrane basin base 108 surrounded by sidewall 106 to form a vessel sufficient to hold water or other liquids.

Some embodiments of cultureware include 2, 3, 4, 6, 12, 24, 96, 384, or 1536-well plates, petri dishes, and T-flasks. A one-well cultureware vessel can be used for culturing an expanse of one type of cell.

Projecting upward from base 108 are fins 104, which are elongated protrusions. Fins 104 are parallel to one another and all at one height, formed from the same gas-permeable material as the rest of the basin base. The height of fins in the exemplary embodiment is at a single, common height between 700 μm and 1000 μm.

Fin heights between 400 μm and 1000 μm, widths between 300 μm and 500 μm, and pitches between 200 μm and 500 μm tend to work well for culturing cells. If the bottom basin and fins are made of gas-permeable silicone rubber, these dimensions allow diatomic oxygen and carbon dioxide to pass through a thin bottom and be channeled through the fins. What also works around this scale is where each one of the fins has a ratio of height to width to pitch of 4-10 to 3-5 to 2-5.

As seen in the geometry legend, a fin can be defined with three axes. An x-axis runs longitudinally along the length of the fin and parallel with an overall bottom plane of the vessel. A y-axis projects laterally and parallel with the bottom plane. A z-axis projects vertically upward, perpendicular to the bottom. A centerline drawn parallel with the z-axis confirms that the fin is symmetric around it.

Each neighboring pair of fins 104 defines a groove 110 that lies between them. Grooves 110 have flat bottoms. In some embodiments, the grooves can have slopes or depressions. The depressions can be in the form of linear trenches, point pits, or otherwise.

Rows of the fins spread across the entire upper surface of basin base 108 except for the perimeter. The rows reach just shy of the walls by a millimeter or so. The parallel fins 104 extend just short of sidewall 106, forming perimeter groove 120. Perimeter groove surrounds the fins on all four sides of sidewall 106. When liquid is in the vessel but not as high as the tops of the fins, the perimeter groove allows liquid to migrate between the fins, like in an ice cube tray.

Fin tops 112 can be seen as rounded in the closeup inset portion of the figure. The lateral cross section (i.e., cut in a plane in the y- and z-axes) of each fin 104 is constant throughout its length. Rounded top 112 has a radius that is half of the width of the fin such that top 112 is entirely rounded.

FIG. 1B shows underneath cultureware 102, exposing elongated footings 114 that project downward from the bottom surface of basin bed 114. Footings 114 are separated by spans 115 and gaps 116. Spans 115 and gaps 116 allow airflow to circulate relatively freely in a thin layer underneath cultureware 102.

Elongated footings 114 on the bottom extend parallel to the long side of cultureware 102, while fins 104 on the top (see FIG. 1A) extend perpendicular to the long side. That is, elongated footings 114 extend laterally with respect to fins 104. Spans 115, which run parallel to the elongated footings, are the empty space underneath grooves and fins. Footings can be positioned with respect to top-side features so that the spans and grooves deform to concentrate cells, as described later on. To that end, the tops of the fins can help errant cells that have settled upon them to fall off into the grooves and depressions.

In some embodiments, fins can extend uninterrupted across the basin base with no gaps. While this may impede some airflow, it may result in more consistent minima when using a resilient membrane (see below).

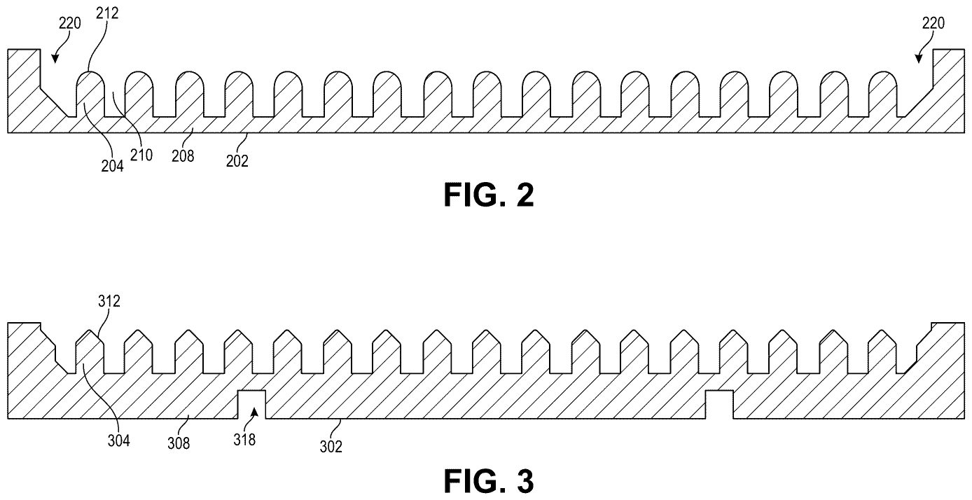

FIG. 2 is a vertical cross section of rounded-top fins in the bottom of cultureware 202. Cultureware 202 is a 24-well plate, and the figure shows the bottom of one well. The cross-section cuts laterally through several fins 204 and basin base 208. The spaces between fins 204 define grooves 210.

At the extreme left and right of the figure are the cross-sections of peripheral groove 220, where it meets the sidewall. The bottom of peripheral groove is at the same depth as that of the between-fin grooves, albeit with a chamfer. In some embodiments, the peripheral grooves'bottom can be deeper or shallower than the depth of the between-fin grooves.

Each fin 204 has rounded top 212. The rounded top is symmetric and has a radius of half of the width of the fin such that top 112 is entirely rounded from side to side. If a cell (or debris) falls onto the rounded top of a fin, it is likely to fall into the adjacent groove, either to the left or the right of the fin, especially if liquid media surrounding the cell is agitated or the cultureware is jiggled. In this way, the cell would join other cells within the groove.

Imaging the cells can be aided by using a transparent material for the basin base. If the grooves are flat bottomed, and the basin base material directly underneath each groove has a constant thickness along a length of the groove, then a microscope light that illuminates the groove can pass through the material (and cells) without excess distortion. In this way, cells can be counted and assessed.

FIG. 3 is a vertical cross section of pointed-top fins in the bottom of cultureware 302. This is also the bottom of one well of a 24-well plate. The cross-section cuts laterally through several fins 304 and basin base 308.

In the bottom surface of basin base 308 are linear notches 318. The notches allow for greater airflow underneath cultureware 302 and add to its flexibility.

Each fin 304 has a gabled top 312. Gable top 312 is symmetric and features slanted sides that descend diagonally from a center point on the top down to its sides. If a cell falls onto the fin, it will likely slide off into an adjacent groove, like rain off of a roof.

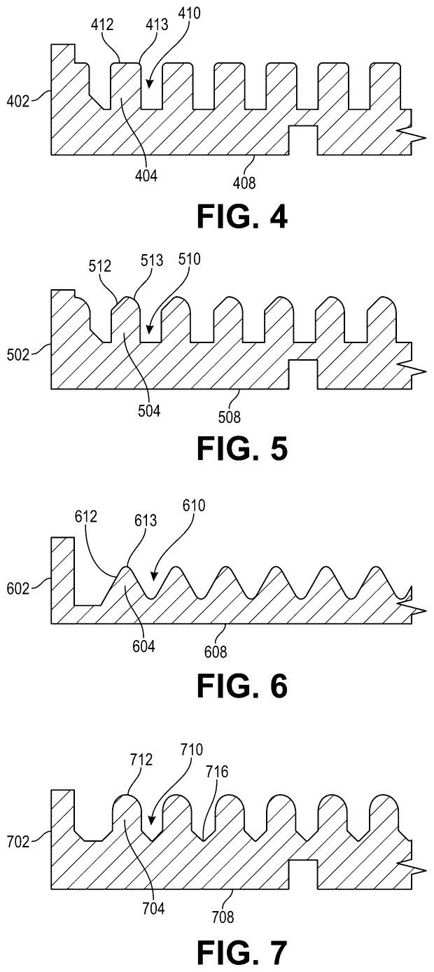

FIG. 4 is a vertical cross section of radius-cornered top fins in the bottom of cultureware 402. Fins 404 project upward from basin base 408. Each fin 404 is symmetric about a center plane and has radiused top 412. Top 412 includes small radius 413 on each edge. The larger the radius, the higher the probability that a cell will slide off of the top into an adjacent groove 410.

FIG. 5 is a vertical cross section of chamfered-and rounded-top fins in the bottom of cultureware 502. Fins 504 project upward from basin base 508, defining grooves 510 between them. Each fin 504 is asymmetric about a center plane. On one side of the top is slanted top 512, and on the other side is rounded with large radius 513. The slant and radius meet at a center point on the top. Slanted top 512 descends diagonally from the center point down to the fin's side. Radius 513 starts at the center point and, having a radius that is half of the width of fin 504, rounds over to the fin's side.

Due to the asymmetry of the top of fin 504, a cell on the top of a fin may be more likely to slide down one side rather than the other, for example, the slanted side instead of the radiused side.

FIG. 6 is a vertical cross section of triangular fins in the bottom of cultureware 602. The cross-section cuts laterally through fins 604, basin base 608, and grooves 610. Each fin 604 includes a heavily slanted top 612, which is symmetric. The slant is at 30° from vertical on both sides.

Each top 612 is crowned by rounded-over peak 613. Each groove 610 includes a rounded, radiused depression. A cell falling onto a fin has a good chance of sliding down into a groove.

FIG. 7 is a vertical cross section of rounded-top fins with pointed bottom grooves in the bottom of cultureware 702. Fins 704 project upward from basin base 708, defining grooves 710 inbetween.

Each fin is crowned by rounded top 712. Meanwhile, each groove has central depression 716, terminating in a point in the figure. The point in the figure represents a linear trench that extends into and out of the page. The trench urges cells into its center, thereby forcing them together.

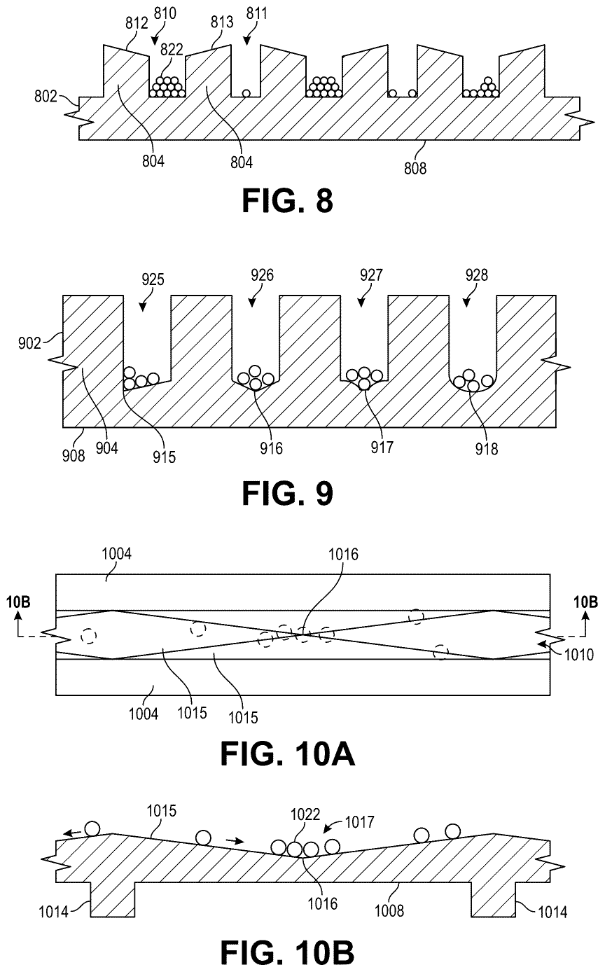

FIG. 8 is a vertical cross section of pairs of fins slanted into common grooves in the bottom of cultureware 802. Fins 804 project upward from basin bottom 808 and define grooves in between. Fins that share a groove are called neighboring fins.

Fin top 812 slopes down to the right, and fin top 813 slopes down to the left. Both slopes run toward common groove 810, which is shared between the pair of fins 804. Each fin is asymmetric about a vertical plane. As shown, cells 822 tend to gather and concentrate in grooves whose adjacent fin tops are slanted toward them. Meanwhile, other grooves 811, whose adjacent fin tops slant away from them, are less likely to gather cells.

The slanted tops can alternate as shown, resulting in alternating “concentration grooves” that concentrate cells. The fin tops can be slanted or rounded in order to direct cells into the concentration grooves. The concentration grooves can have flat bottoms, as shown, or other geometries that assist in concentrating cells.

Grooves that are not concentration grooves, called non-preferred grooves, may have depressions or bottom geometries that deal with lower concentrations of cells. Any groove may have depression features that enhance the concentration of cells.

FIG. 9 is a vertical cross section of several types of groove bottoms of cultureware 902. Fins 904 project upward from basin base 908 to form grooves in between them.

Groove 925 has a simple diagonally slanted bottom, which defines linear trench depression 915 along its left side. Groove 925 is asymmetric. Cells will naturally fall into trench depression 915 on one side. Other designs may urge cells to concentrate in a middle of a groove.

Groove 926 is a V-shaped, dual diagonal slanted bottom, which defines a central linear trench depression 916 in the groove's middle. This can keep cells away from the tall sides of the fins.

Groove 927 is a dual convex radius bottom, which defines a Y-like central linear trench depression 917 in the groove's middle. Like the bottom of groove 926, this can keep cells away from the tall sides of the fins.

Groove 928 is a dual concave radius bottom, which defines a U-shaped central half-pipe depression 918 in the groove's middle.

A technical advantage of symmetric grooves is that cells in the center may be easier for an end user to wash out for harvesting. A symmetric groove may be simpler to model for oxygen permeation and distribution. It may also be simpler to manufacture at such small scales.

Instead of linear trench depressions, some grooves can have point-like depressions that concentrate cells in two dimensions.

FIGS. 10A-10B illustrate a top view and cross section of a shallow diamond point groove in the bottom of a cultureware. Neighboring fins 1004 project up from basin base 1008 to define common groove 1010. Footings 1014 are integral to basin base 1008 material and project down from it.

Surfaces 1015 of basin base 1008 within groove 1010 slant downward to meet at low point 1016. The surfaces and point define depression 1017. Low point 1016 of depression 1017 is centered between pair of footings 1014.

The exemplary surfaces are shown as flat and faceted, like a shallow diamond point, but smooth curves are also envisioned. The depression's lowest point can be off center from the footings and/or off to one side of the groove. The depression can be sharper or shallower, depending on cell sizes and desired concentrations.

FIG. 10B illustrates biological cells 1022 rolling or otherwise migrating to low point 1016 in depression 1017. FIG. 10A shows the cells in broken line to avoid obstructing the surfaces. Seed cells congregate near the low point from other parts of the groove and may be more ready to grow and multiply than if they were scattered about.

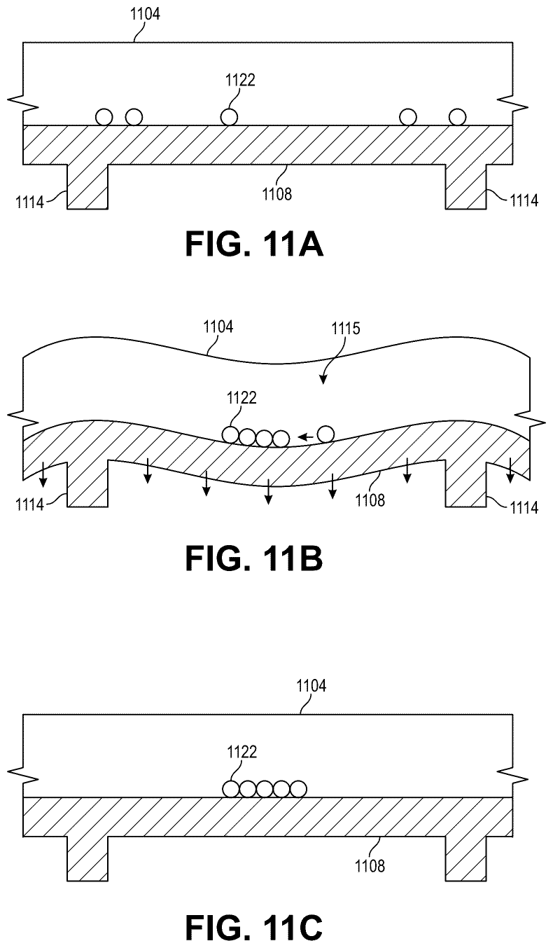

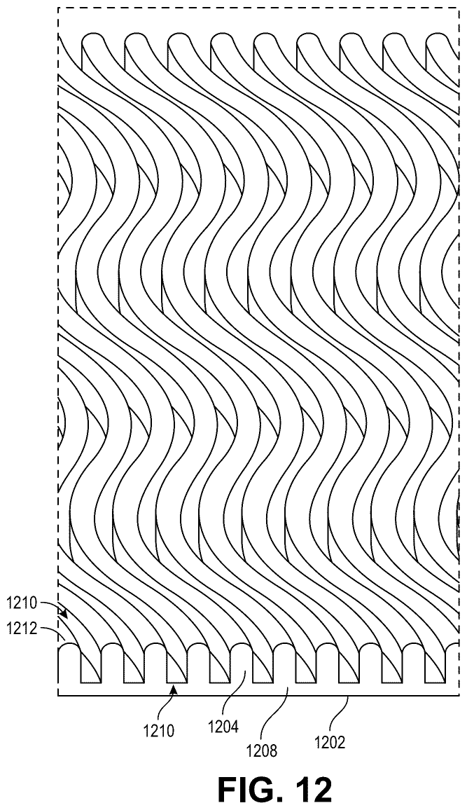

FIGS. 11A-11C are vertical cross section views of a groove of a cultureware. A “cross section view” includes a cross section through the center of a groove along with a fin in the background, for context. Fin 1104 projects upward from basin base 1108. Lateral footings 1114 project downward from the bottom surface of basin base 1108. The fins and base are made of a resilient material that bends under pressure or centripetal loads.

FIG. 11A shows biological cells 1122 dispersed in the groove, sitting where they initially fell from being seeded in an initial liquid medium.

The cultureware is then placed on a centrifuge plate in a centrifuge machine. The plate, and everything that is on it, is spun up so that it experiences an (apparent gravity) g-force equal-to-or-greater-than 10 times a force of gravity. The g-force is sometimes referred to as a Relative Centrifugal Force (RCF). An RCF of over 100 g's is common in centrifuges.

FIG. 11B illustrates the device and cells undergoing centrifugal loads in a centrifuge machine. Compliant fin 1104 and basin bottom 1108 deform to create local minimum 1115, the lowest point of which is centered between lateral footings 1114. Cells 1122, under their own weight, move down the depression well to congregate at the lowest point, i.e. local minimum 1115. This clustering of cells occurs wherever there are local minima in the grooves. The device is then removed from the centrifuge machine.

FIG. 11C illustrates fin 1104 and basin base 1108 returned back to their original form. Cells 1122, however, remain clustered. The cells may stay there due to friction, a small amount of stickiness with each other, or simply because no external force moves them. The cells can then be cultured more readily from their concentrated clusters than if they were dispersed.

FIG. 12 is a top-side perspective view of the bottom of cultureware 1202. Parallel fins 1212 project upward from basin base 1208, defining grooves 1210. Each fin top 1212 is fully round.

Fins 1204 are in rows that undulate laterally in a sinusoidal, snakelike fashion. Although the fins undulate laterally, they do not touch each other and are considered parallel. Like the parallel fins, grooves 12010 undulate laterally back and forth in soft curves. The fins and grooves do not need to be as curvy as shown in the figure.

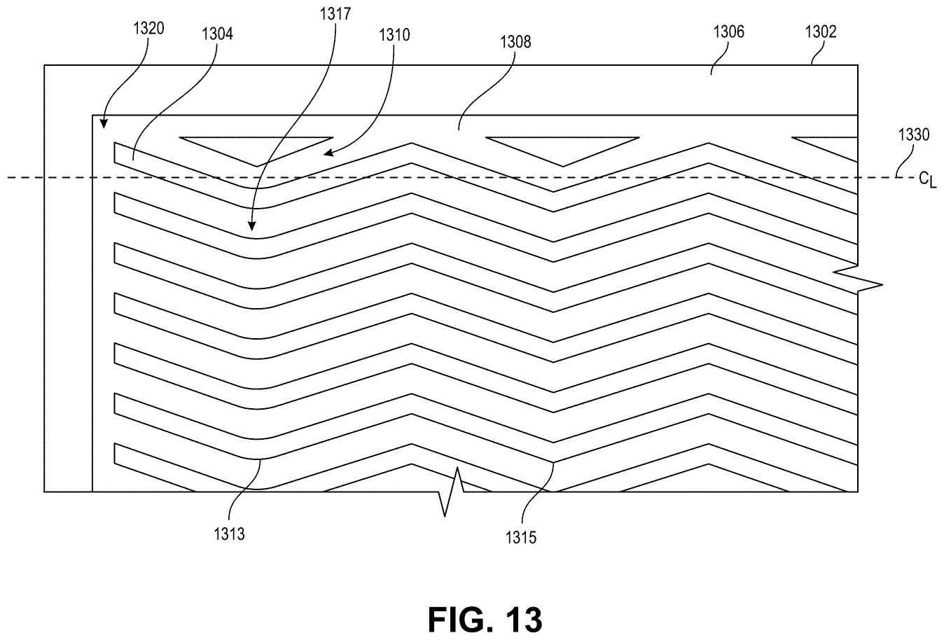

FIG. 13 is a top view of a zig-zag pattern of rows of fins in a bottom of cultureware 1302. Fins 1304 project upward (out of the page) from basin bottom 1308, defining grooves 1310 between them.

Peripheral groove 1320 surrounds fins 1304 and follows sidewall 1306 all of the way around the perimeter of cultureware 1302.

Each fin 1304 undulates symmetrically around average centerline 1330. That is, zigs in one direction (upwards on the page) are equaled by zags in the opposite direction (downward on the page). The zig-zag pattern can be considered symmetric with its upside-down counterpart. Each grooves 1310 also is symmetric around an average centerline.

An exception to the symmetry around centerline 1330 is in some inflection points of the fins, where the rows change direction. A fin is rounded at rounded inflection point 1313, with a slight radius. In contrast, the same fin is pointed at pointed inflection point 1315. Rounded and pointed inflection points may be used for concentrating different types of cells. Overall, the entirety of the fin is mostly symmetric with respect to an average centerline.

The grooves are where cells will settle. Tilting cultureware 1302 or 1202 (see FIG. 12) can move cells such that they congregate in concave portions 1317 of the grooves. That is, the cells will concentrate in low points with respect to gravity and trapped by a V- or U-shapes in the fins. Tilting can be done by an operator, a robot, or on a static surface.

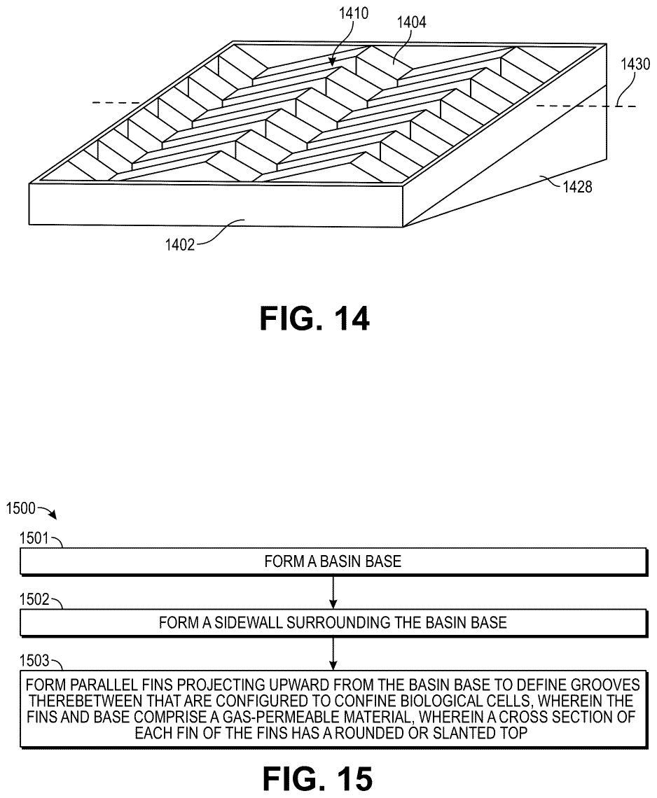

FIG. 14 is a perspective view of an undulating fin cultureware 1402 on a pedestal wedge. In the figure, the sidewall is shortened and fins greatly exaggerated for clarity. In cultureware 1402, fins 1404 project upward from a basin base and define grooves 1410 in between.

Fins 1404 zig-zag laterally within the cultureware. The fins zig-zag around average centerline. Average centerline 1430 of a fin row is perpendicular to a slope of wedge 1428. Cells within the grooves will gravitate toward concave V-shaped portions of the fins, concentrating there. The assembly of the wedge and cultureware can be left overnight for cells to have time to slide and move toward the low points, or it can be subject to a centrifuge so as to expedite migration of the cells.

The average centerline does not need to be perpendicular but can generally extend non-parallel to a slope of the wedge.

FIG. 15 is a flowchart illustrating process 1500 in accordance with an embodiment. In operation 1501, a basin base is formed. In operation 1502, a sidewall surrounding the basin base is formed. In operation 1503, parallel fins that project upward from the basin base are formed to define grooves therebetween that are configured to confine biological cells. The fins and based comprise a gas-permeable material. A cross section of each fin of the fins has a rounded or slanted top. The forming operations can be performed all at one time or at separate times and in various orders

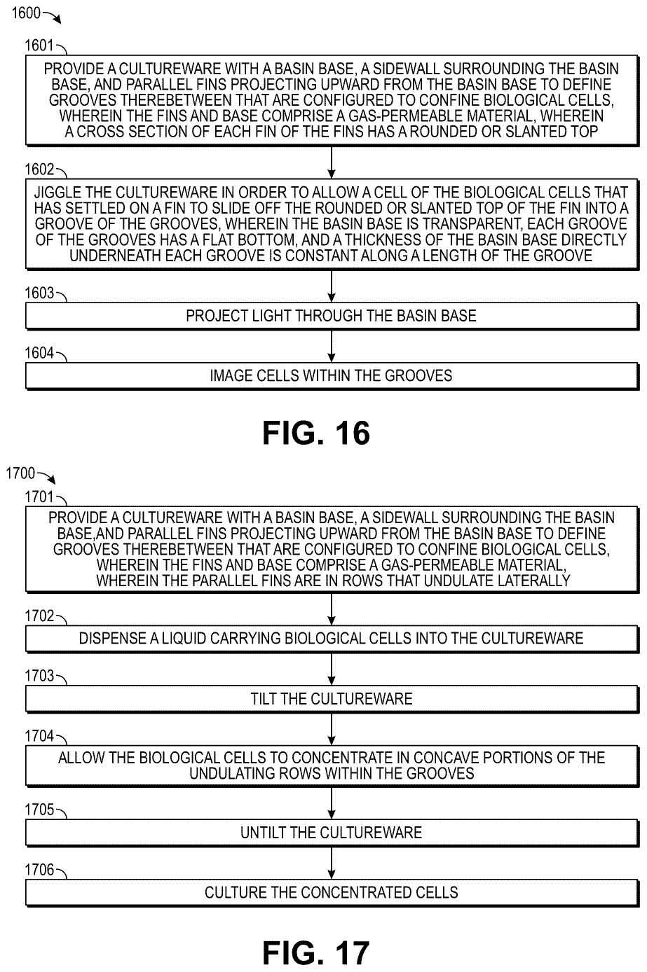

FIG. 16 is a flowchart illustrating process 1600 in accordance with an embodiment. In operation 1601, a cultureware is provided that has a basin base, a sidewall surrounding the basin base, and parallel fins projecting upward from the basin base to define grooves therebetween that are configured to confine biological cells. The fins and base are made from a gas-permeable material. A cross section of each fin of the fins has a rounded or slanted top. In operation 1602, the cultureware is jiggled, or a liquid medium agitated, in order to allow a cell of the biological cells that has settled on a fin to slide off of the rounded or slanted top of the fin into a groove of the grooves. The basin base is transparent, each groove of the grooves has a flat bottom, and a thickness of the basin base directly underneath each groove is constant along a length of the groove. In operation 1603, light is projected through the basin base. In operation 1604, cells within the grooves are imaged.

FIG. 17 is a flowchart illustrating process 1700 in accordance with an embodiment. In operation 1701, a cultureware is provided that has a basin base, a sidewall surrounding the basin base, and parallel fins projecting upward from the basin base to define grooves therebetween that are configured to confine biological cells. The fins and base can be made of a gas-permeable material. The parallel fins can be in rows that undulate laterally. In operation 1702, a liquid carrying biological cells can be dispensed into the cultureware. The cells disperse randomly. In operation 1703, the cultureware is tilted. In operation 1704, the biological cells are allowed to move and concentrate in concave portions of the undulating rows within the grooves. In operation 1705, the cultureware is righted (i.e., untilted with respect to gravity). In operation 1706, the concentrated cells are cultured.

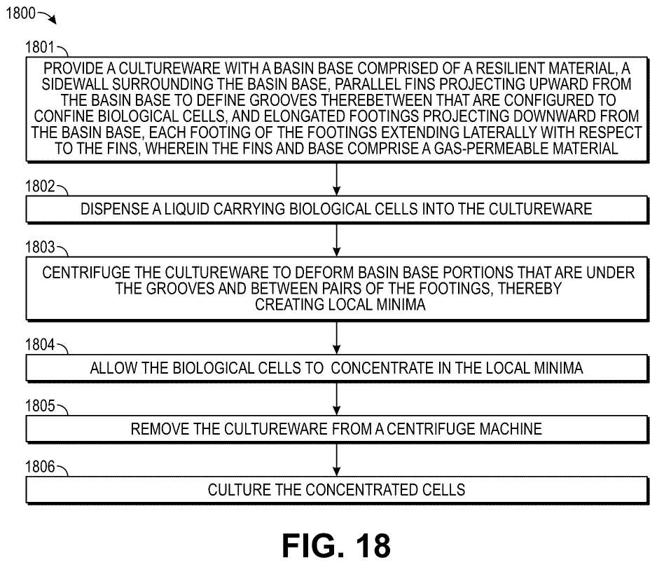

FIG. 18 is a flowchart illustrating process 1800 in accordance with an embodiment. In operation 1801, a cultureware is provided that has a basin base comprised of a resilient material, a sidewall surrounding the basin base, parallel fins projecting upward from the basin base to define grooves therebetween that are configured to confine biological cells, and elongated footings projecting downward from the basin base, each footing of the footings extending laterally with respect to the fins. The fins and base are made of a gas-permeable material. In operation 1802, a liquid carrying biological cells is dispensed into the cultureware. In operation 1803, the cultureware is centrifuged or pressurized in order to deform basin base portions that are under the grooves and between pairs of the footings, thereby creating local minima. In operation 1804, the biological cells are allowed to move and concentrate in the local minima. In operation 1805, the cultureware is removed from a centrifuge machine. In operation 1806, the concentrated cells are cultured.

### Enhanced Post-Electroporation Cell Recovery Using a High-Density Cell Respirator

Electroporation is a powerful tool for vector-free delivery of nucleic acids and gene-editing components into cells. However, the process imposes substantial biophysical stress on cells through transient membrane permeabilization, ionic imbalance, and metabolic disruption.

It was hypothesized that the HDCR cultureware microenvironment, characterized by enhanced oxygenation, shear-free conditions, and close cell-cell proximity, would improve post-electroporation recovery of stressed cells and enhance gene expression relative to conventional polystyrene flask cultureware.

To test the hypothesis, (Human Embryonic Kidney) HEK293T cells were electroporated with a blue fluorescent protein (BFP) plasmid using a Lonza 4D-Nucleofector® X Unit across a range of seeding densities. The seeding densities spanned 2M, 4M, 6M, 8M, and 10M (million) cells per 2 cm² well of a 24-well HDCR plate, in accordance with an embodiment, or per T-75 (i.e., 75 cm²) polystyrene flask of the prior art. All had 2 μg of deoxyribonucleic acid (DNA) per 1M cells. For each condition, cells were electroporated in a single cuvette at twice the target density and evenly divided post-electroporation between the 24-well HDCR plate and the T-75 flask. Cells were recovered for 24 hours, then harvested and analyzed by flow cytometry following Annexin V/propidium iodide staining to quantify apoptosis and BFP mean fluorescence intensity (MFI).

Flow cytometric analysis revealed enhanced post-electroporation recovery in the HDCR embodiments relative to T-75 flasks, at least at lower seeding densities. At 2M cells, HDCR cultures achieved approximately 1.7-fold higher viability than polystyrene (83.3% vs ~50%), with a sustained ~1.6-fold advantage at 4M cells. In contrast, the polystyrene cultures exhibited improved viability only at higher seeding density, reaching ~60% live cells at 6M cells, where viability converged with HDCR. This suggests that cell density plays an important role in cell recovery. Non-viable populations across conditions comprised mixed apoptotic, necrotic, and necroptotic states. Consistent with improved recovery, BFP⁺ cell MFI was higher in HDCR than in polystyrene across all densities, with fold increases ranging from 1.3 to 4.6.

These findings suggest that the HDCR microenvironment supports improved survival and functional recovery of electroporated cells, consistent with prior observations of increased viral titer. More broadly, as electroporation remains a well-established approach across cell and gene therapy workflows, strategies that optimize post-electroporation recovery represent a significant opportunity to improve process efficiency, robustness, and translational scalability across the Cell and Gene Therapies (CGT) field.

A method for improved deoxyribonucleic acid (DNA) or ribonucleic acid (RNA) transfer can include electroporating cells sufficient to introduce foreign material from outside biological cells into to the cells. The foreign material can include DNA or RNA.

The method can further include seeding the cells into a cultureware device. The cultureware can include a basin base, a sidewall surrounding the basin base, and parallel fins projecting upward from the basin base and defining grooves therebetween configured to confine biological cells, wherein the fins and base comprise gas-permeable material. A cross section of each fin of the fins can have a rounded or slanted top, and/or the parallel fins can be in rows that undulate laterally, and/or there may be elongated footings projecting downward from the basin base, each footing of the footings extending laterally with respect to the fins, wherein the basin base is comprised of a resilient material. The parallel fins can extend short of the sidewall to form a perimeter groove along the sidewall that surrounds the fins.

The cells can be seeded at 2 million or fewer cells per cm² in the cultureware device. The cells can include apoptotic, necrotic, and necroptotic cells along with viable cells.

The cells are then left to recover. The cells may expand (multiply), express gene product, not express product that would otherwise be expressed before electroporation, or otherwise.

Alternatively on another device, electroporated cells can be seeded on a device with cell concentration features at a rate between 1 and 2 million cells per cm², then left to recover.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” in reference to a temperature or other engineering units includes measurements or settings that are within ±1%, ±2%, ±5%, ±10%, or other tolerances of the specified engineering units as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A cultureware apparatus comprising:
a basin base;
a sidewall surrounding the basin base; and
parallel fins projecting upward from the basin base and defining grooves therebetween configured to confine biological cells,
wherein the fins and base comprise a gas-permeable material;
wherein a cross section of each fin of the fins has a rounded or slanted top.

2. The apparatus of claim 1 wherein a lateral cross section of each fin is symmetric around a vertical line.

3. The apparatus of claim 1 wherein each top is entirely slanted or entirely rounded.

4. The apparatus of claim 1 wherein an edge of each top is chamfered or filleted.

5. The apparatus of claim 1 wherein a pair of adjacent fins each have a top in which more than a half of the respective top slopes toward a groove shared between the pair.

6. The apparatus of claim 1 wherein the parallel fins extend short of the sidewall to form a perimeter groove along the sidewall that surrounds the fins.

7. The apparatus of claim 1 wherein the parallel fins are in rows that undulate laterally.

8. The apparatus of claim 7 wherein the undulating rows are in a wavy pattern or a zig-zag pattern.

9. The apparatus of claim 8 wherein inflection points of the zig-zag pattern are rounded.

10. The apparatus of claim 7 wherein each fin in the undulating rows is symmetric about an average centerline.

11. A cultureware assembly comprising:
a pedestal wedge; and
the apparatus of claim 1 supported underneath by the wedge with an average centerline of the rows extending non-parallel to a slope of the wedge.

12. The apparatus of claim 1 wherein:
the basin base is transparent;
each groove of the grooves has a flat bottom; and
a thickness of the basin base directly underneath each groove is constant along a length of the groove.

13. The apparatus of claim 1 wherein the basin base includes a depression within a groove of the grooves.

14. The apparatus of claim 13 wherein the depression narrows to a point.

15. The apparatus of claim 13 wherein the depression includes a trench.

16. The apparatus of claim 1 further comprising:
elongated footings projecting downward from the basin base, each footing of the footings extending laterally with respect to the fins

17. The apparatus of claim 16 wherein the basin base includes a depression within a groove of the grooves, the depression centered between a pair of the footings.

18. The apparatus of claim 1 wherein at least one of the fins has a height between 400 μm and 1000 μm, a width between 300 μm and 500 μm, and a pitch between 200 μm and 500 μm.

19. (canceled)

20. A cultureware apparatus comprising:
a basin base;
a sidewall surrounding the basin base; and
parallel fins projecting upward from the basin base and defining grooves therebetween configured to confine biological cells,
wherein the fins and base comprise a gas-permeable material;
wherein the parallel fins are in rows that undulate laterally.

21-25. (canceled)

26. A cultureware apparatus comprising:
a basin base comprised of a resilient material;
a sidewall surrounding the basin base;
parallel fins projecting upward from the basin base and defining grooves therebetween configured to confine biological cells; and
elongated footings projecting downward from the basin base, each footing of the footings extending laterally with respect to the fins,
wherein the fins and base comprise a gas-permeable material.

27-43. (canceled)
